(19) United States
(12) Patent Application Publication
(10) Pub. No.: US 2022/0313144 A1
(43) Pub. Date: Oct. 6, 2022

(54) SYSTEM AMD METHOD FOR DETERMINING TREATMENT OUTCOMES FOR NEUROLOGICAL DISORDERS BASED ON FUNCTIONAL CONNECTIVITY PARAMETERS

(71) Applicant: The General Hospital Corporation, Boston, MA (US)

(72) Inventors: Eric S. Rosenthal, Boston, MA (US); Daniel B. Rubin, Boston, MA (US)

(21) Appl. No.: 17/642,167

(22) PCT Filed: Sep. 14, 2020
(86) PCT No.: PCT/US2020/050683

§ 371 Date: Mar. 10, 2022

Related U.S. Application Data
(60) Provisional application No. 62/899,413, filed on Sep. 12, 2019.

(51) Int. Cl. A61B 5/00; A61B 5/369; G16H 50/30

(52) U.S. Cl. CPC A61B 5/4076; A61B 5/369; A61B 5/7267; G16H 50/30; A61B 5/742

(57) ABSTRACT

A system for determining a treatment outcome for a subject for a neurological disorder includes a processor, a classifier and a display. The processor is configured to receive a set of electroencephalogram (EEG) data associated with the subject and to determine at least one functional connectivity parameter based on the EEG data. The classifier is coupled to the processor and is configured to receive the at least one functional connectivity parameter and to generate a prediction for a treatment outcome based on the at least one functional connectivity parameter. The display is coupled to the classifier and is configured to display the prediction for the treatment outcome.

### CROSS-REFERENCE TO RELATED APPLICATIONS

This application is based on, claims priority to, and incorporates herein by reference in its entirety U.S. Ser. No. 62/899,413 filed Sep. 12, 2019, and entitled “Status Epilepticus Anesthesia Wean Assist Tool (SEAWEAT).”

### STATEMENT REGARDING FEDERALLY SPONSORED RESEARCH OR DEVELOPMENT

This technology was made with government support under grants R25NS065743, R01NS062092, K24NS088568 and NS105950 awarded by the National Institutes of Health. The government has certain rights in the technology.

### FIELD

The present disclosure relates generally to systems and methods for monitoring a subject and, more particularly, to systems and methods for determining a treatment outcome for neurological disorders and injuries based on functional connectivity parameters.

### BACKGROUND

Management of treatments for neurological disorders using, for example, anesthesia or anti-epileptic drugs (AEDs), presents numerous challenges. For example, refractory status epilepticus (RSE) is a common and highly morbid condition characterized by a state of continuous or recurrent seizures despite adequate treatment with conventional anti-epileptic drugs. Failure to adequately control seizures can result in long term neurologic sequelae or death. The current standard of care for refractory status epilepticus involves treatment with intravenous (IV) anesthesia (e.g., an intravenous third-line anesthetic agent (IV-TLA)) to cease seizure activity through production of a medically-induced coma. Intravenous anesthetics are typically titrated in refractory status epilepticus to achieve either seizure suppression or a burst suppression pattern on continuous electroencephalography (cEEG). However, an optimum treatment paradigm, for example, the appropriate duration of treatment with IV anesthetics or the appropriate timing of weaning of anesthetics, is unknown. Patients are routinely maintained in a medically-induced coma with a continuous infusion of anesthetics for one day or more prior to weaning anesthetics, but this duration is arbitrary. Many patients may be able to be safely weaned from IV anesthesia sooner. Additionally, periodic and rhythmic EEG patterns on the ictal-interictal continuum (IIC) may emerge during anesthetic weaning, prompting resumption of IV-TLA therapy despite their unknown significance.

Though the use of IV anesthetics is necessary for treatment of RSE, the prolonged use of anesthetics increases the risk of treatment-associated adverse effects. Substantial morbidity associated with RSE is attributable to the prolonged use of anesthetics such as IV-TLA. Accordingly, interventions that could minimize the duration of anesthesia have the potential to improve outcomes. Prolonged duration of IV-TLA therapy can expose patients to the risk of ventilator-associated pneumonia, and IV-TLAs themselves are associated with hypotension, cardiac arrhythmias, hepatotoxicity, and other agent-specific sequelae, such as Propofol infusion syndrome. Periods of prolonged immobility in the intensive care unit also incur the associated risks of deep vein thromboses, deconditioning, and healthcare associated infections. However, prematurely weaning anesthetics exposes patients to risks from inadequately treated status epilepticus including the risk of recurrence of seizures. Persistent seizure activity and other ictal-interictal continuum (IIC) EEG patterns have been shown to lead to hyperglycolysis, elevated brain tissue lactate, brain tissue hypoxia, and long-term structural changes in the brain, which carry the risk of long-term neurologic sequelae. Persistent status epilepticus is also associated with a number of adverse systemic effects including hypoxia, cardiac dysfunction, renal failure, and metabolic derangements.

In another example, seizures and ictal-interictal continuum (IIC) activity may impact recovery and are a cause of prolonged depressed level of consciousness following acute brain injury (ABI). Empiric AED treatment for electrophysiological activity for IIC of uncertain significance is controversial and challenging to evaluate. Given heterogeneity in presenting structural neurologic deficits, variable pharmacodynamics, and potential sedative effects of AEDs, there is risk that harm of empiric treatment may outweigh the benefit in many patients. Encephalopathic patients with concerning IIC pattern EEG findings are commonly trialed on escalating doses of AEDs, often with minimal benefit, whereas potentially beneficial AED therapy may be withheld from other patients with qualitatively more benign-appearing EEG patterns.

It would be desirable to provide a system and method to determine or predict treatment outcomes for neurological disorders such as RSE and disorders of consciousness after ABI for which treatment requires the use of anesthesia or AEDs Such a system and method may be used to guide or assist clinicians with managing treatment, for example, safely minimizing the use of anesthesia or AEDs, and improve patient outcome.

### SUMMARY

In accordance with an embodiment, a system for determining a treatment outcome for a subject for a neurological disorder includes a processor, a classifier and a display. The processor is configured to receive a set of electroencephalogram (EEG) data associated with the subject and to determine at least one functional connectivity parameter based on the EEG data. The classifier is coupled to the processor and is configured to receive the at least one functional connectivity parameter and to generate a prediction for a treatment outcome based on the at least one functional connectivity parameter. The display is coupled to the classifier and is configured to display the prediction for the treatment outcome.

In accordance with another embodiment, a method for determining a treatment outcome for a subject for a neurological disorder includes acquiring a set of electroencephalogram (EEG) data from the subject, determining, using a processor, at least one functional connectivity parameter based on the set of EEG data, generating, using a classifier, a prediction for a treatment outcome based on the at least one functional connectivity parameter and displaying the prediction for the treatment outcome using a display.

### BRIEF DESCRIPTION OF THE DRAWINGS

The present disclosure will hereafter be described with reference to the accompanying drawings, wherein like reference numerals denote like elements.

FIG. 1A is a schematic block diagram of an example physiological monitoring system in accordance with an embodiment;

FIG. 1B is a schematic block diagram of an example physiological monitoring system in accordance with an embodiment;

FIG. 2 is an example physiological monitoring system in accordance with an embodiment;

FIG. 3 is a schematic block diagram of a system for determining a treatment outcome for neurological disorders based on functional connectivity parameters in accordance with an embodiment;

FIG. 4 illustrates a method for determining a treatment outcome for neurological disorders based on functional connectivity parameters in accordance with an embodiment;

FIG. 5 illustrates a method for determining a treatment outcome of weaning of anesthesia in status epilepticus based on functional connectivity parameters in accordance with an embodiment;

FIG. 6 illustrates a method for training a classifier for determining a treatment outcome of weaning of anesthesia in status epilepticus based on functional connectivity parameters in accordance with an embodiment;

FIG. 7 illustrates a method for determining an outcome of treatment of a subject after acute brain injury with an antiepileptic drug based on functional connectivity parameters in accordance with an embodiment;

FIG. 8 illustrates a method for training a classifier for determining an outcome of treatment of a subject after acute brain injury with an antiepileptic drug based on functional connectivity parameters in accordance with an embodiment;

FIG. 9 illustrates a method for calculating functional connectivity from cEEG signals in accordance with an embodiment;

FIG. 10 shows graphs illustrating a comparison of frequency-based parameters (or metrics) between successful and unsuccessful anesthesia weans in accordance with an embodiment;

FIG. 11 shows graphs illustrating a comparison of functional connectivity parameters (or metrics) between successful and unsuccessful anesthesia weans in accordance with an embodiment;

FIGS. 12A and 12B are graphs illustrating accuracy testing of a classifier in accordance with an embodiment;

FIG. 13A is a graph illustrating mean GCS score trajectories over 100 bootstrap iterations during training of a classifier with five-fold internal cross-validation in accordance with an embodiment;

FIG. 13B is a graph illustrating mean GCS score trajectories generated by a trained classifier in accordance with an embodiment; and

FIG. 13C is a graph illustrating mean GCS score trajectories for a control model for evaluating performance of a classifier in accordance with an embodiment.

### DETAILED DESCRIPTION

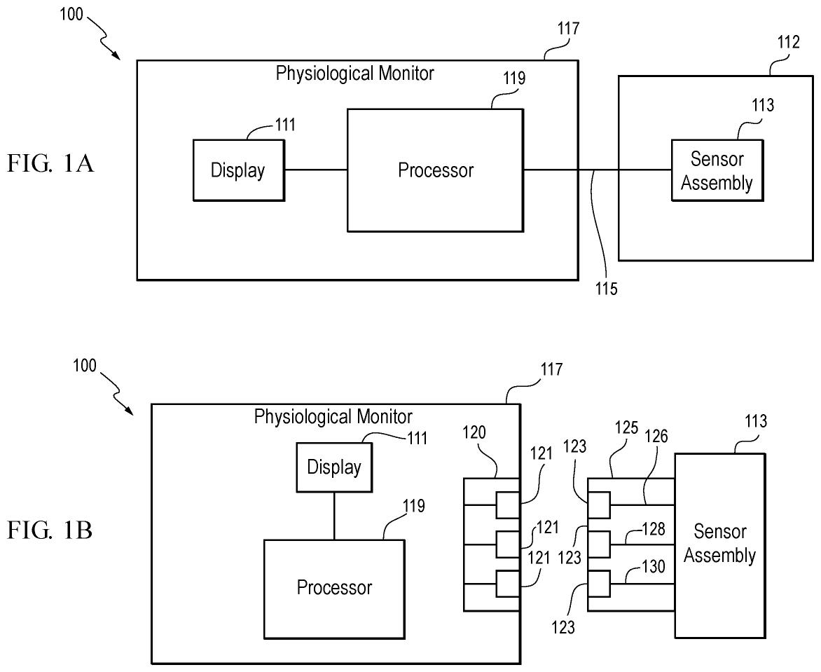

FIGS. 1A and 1B illustrate example monitoring systems and sensors that can be used to provide monitoring of a subject, for instance, during administration anesthesia, sedation, or other medical procedure. For example, FIG. 1A shows an embodiment of a monitoring system 100. Specifically, using monitoring system 100, a subject 112 is monitored using a sensor assembly 113 included therein, which can transmit various signals over a cable 115 or other communication link or medium to a physiological monitor 117. The physiological monitor 117 includes a processor 119 and, optionally, a display 111 for reporting a variety of information, such as information related to the condition of the subject 112, state of consciousness or transitions from states of consciousness of the subject 112, as well as other states of the subject 112. In some designs, the monitoring system 100 may be a portable monitoring system. In other designs, the monitoring system 100 may be a pod without a display, and adapted to provide physiological parameter data to a display.

The sensor assembly 113 can include one or more sensing elements such as, for example, electrical EEG sensors, or the like. The sensor assembly 113 can generate respective signals by measuring physiological parameters of the subject 112. The signals are then processed by one or more processors 119. The one or more processors 119 then communicate the processed signal to the display 111, if provided, or other logically connected output. In an embodiment, the display 111 is incorporated in the physiological monitor 117. In another embodiment, the display 111 is separate from the physiological monitor 117.

For clarity, a single block is used to illustrate the sensor assembly 113 shown in FIG. 1A. However, it should be understood that the sensor assembly 113 shown is intended to represent one or more sensors configured for placement at a variety of locations about the skull of the subject 112 and acquire various physiological signals. In one embodiment, the sensor assembly 113 can include sensors of one type, such as EEG sensor. In other embodiments, the sensor assembly 113 can include multiple types of sensors, such as EEG sensors, brain oxygenation sensors, optical sensors, galvanic skin response sensors, and so on. In each of the foregoing embodiments, additional sensors of different types are also optionally included. Other combinations of numbers and types of sensors are also suitable for use with the monitoring system 100.

In some embodiments of the system shown in FIG. 1A, all of the hardware used to receive and process signals from the sensors are housed within the same housing. In other embodiments, some of the hardware used to receive and process signals is housed within a separate housing. In addition, the physiological monitor 117 of certain embodiments includes hardware, software, or both hardware and software, whether in one housing or multiple housings, used to receive and process the signals transmitted by the sensor assembly 113.

As shown in FIG. 1B, the sensor assembly 113 can include a cable 125. The cable 125 can include three conductors, for example, within an electrical shielding. One conductor 126 can provide power to a physiological monitor 117, one conductor 128 can provide a ground signal to the physiological monitor 117, and one conductor 130 can transmit signals from one sensor in the sensor assembly 113 to the physiological monitor 117. For multiple sensors, one or more additional cables 125 can be provided.

In some embodiments, the ground signal is an earth ground. In other embodiments, the cable assembly 125 may include a fourth conductor (not shown) that can provide a subject reference, sometimes referred to as a subject reference signal, a return, or a subject return, from the sensor assembly 113 to the physiological monitor 117. In some embodiments, the cable 125 carries two conductors within an electrical shielding layer, and the shielding layer acts as the ground conductor. Electrical interfaces 123 in the cable 125 can enable the cable to electrically connect to electrical interfaces 121 in a connector 120 of the physiological monitor 117. In another embodiment, the sensor 113 and the physiological monitor 117 communicate wirelessly.

Processor 119 may be configured to perform a number of steps for processing and analyzing data received from the sensor assembly 113. In particular, processor 119 can be configured to assemble the data in any number of forms, including waveforms, spectrograms, coherograms, modulograms, and so on. In an embodiment, processor 119 can also be configured to determine quantitative parameters of the EEG data (e.g., functional connectivity parameters) received from the sensor assembly 113, as discussed further below. In another embodiment, the processor 119 may also include a classifier that may be used to determine (or predict) a treatment outcome based on at least functional connectivity parameters as discussed further below. Additionally, processor 119 may also be configured to identify other signal markers or signatures associated with the received data using various analysis methods, including waveform analyses, spectral analyses, frequency analyses, coherence analyses and so on. For example, signal markers or signatures can include various signal amplitudes, phases, frequencies, power spectra, frequency distributions, spatial distribution, and so forth. In some configurations, the systems shown in FIGS. 1A and 1B may further include a memory, database or other data storage locations (not shown), accessible by processor 119, to include reference information or other data.

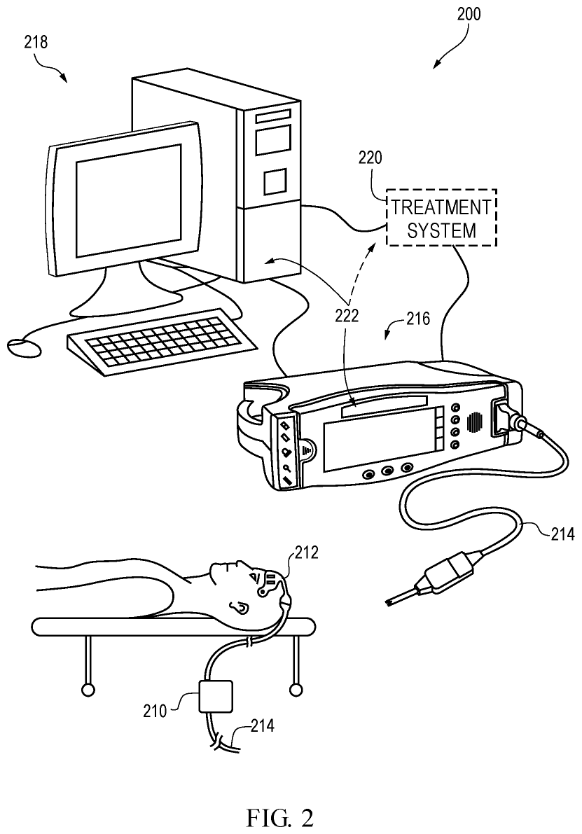

Specifically now referring to FIG. 2, an example system 200 in accordance with the present disclosure is illustrated, for use in monitoring and/or controlling a state of a subject during a medical procedure, or as result of an injury, pathology or other condition. In some aspects, the system 200 could be used to guide or control, as non-limiting examples, medically-induced coma, anesthesia, or sedation.

The system 200 includes a subject monitoring device 212 that may include multiple physiological sensors, such as EEG sensors. However, it is contemplated that the subject monitoring device 212 may incorporate other sensors including blood oxygenation sensors, temperature sensors, acoustic respiration monitoring sensors, galvanic skin response sensors and so forth.

The subject monitoring device 212 is connected via a cable 214 to communicate with a monitoring system 216, which may be a portable system or device, and provides input of physiological data acquired from a subject to the monitoring system 216. Alternatively, the cable 214 and similar connections can be replaced by wireless connections between components. The monitoring system 216 may be configured to receive raw signals acquired by the sensors and assemble, and even display, the raw or processed signals or information derived therefrom.

As illustrated in FIG. 2, the monitoring system 216 may be further connected to a dedicated analysis system 218. In some aspects, the analysis system 218 may receive the data from the monitoring system 216, and, as discussed further below, determine quantitative parameters of the EEG data (e.g., functional connectivity parameters) received from the monitoring system 216. In another embodiment, the analysis system 218 may also include a classifier that may be used to determine (or predict) a treatment outcome based on at least functional connectivity parameters a discussed further below. The analysis system 218 may also generate a report, for example, as a printed report or, preferably, a real-time display of, for example, the quantitative parameters and prediction of treatment outcome. In some aspects, the subject monitoring device 212 may be in communication with a portable processing system 210, which may be configured for perform any number of processing steps, such as identifying and/or relaying information relating to brain states of a subject. Although shown as separate systems in FIG. 2, it is also contemplated that components and/or functionalities of monitoring system 216, analysis system 218 and system 200 may be combined or integrated.

In some configurations, the system 200 may also include a treatment system 220. The treatment system 220 may be coupled to the analysis system 218 and monitoring system 216, such that the system 200 forms a closed-loop monitoring and control system. Such a closed-loop monitoring and control system may be capable of a wide range of operation, and may include a user interface 222, or user input, to allow a user to configure the closed-loop monitoring and control system, receive feedback from the closed-loop monitoring and control system, and, if needed reconfigure and/or override the closed-loop monitoring and control system.

In some configurations, the treatment system 220 may include a drug delivery system not only able to control the administration of anesthetic compounds for the purpose of placing the subject in a state of reduced consciousness influenced by the anesthetic compounds, such as general anesthesia or sedation, but can also implement and reflect systems and methods for bringing a subject to and from a state of greater or lesser consciousness. Other treatments may be administered or facilitated by the treatment system 220 as well.

The present disclosure describes a system and method for determining (or predicting) treatment outcomes based on functional connectivity parameters. Functional connectivity is an analytic schema which describes the structure of activity in the spatial domain. Functional connectivity characterizes global features of higher-order spatial correlations, capturing changes in network activity that would otherwise be invisible by standard review. The system and method may be used to determine or predict treatment outcomes for neurological disorders such as RSE and disorders of consciousness after ABI for which treatment requires the use of anesthesia or AEDs. In an embodiment, the functional connectivity parameters are determined by performing quantitative analysis on the electrophysical signal recorded from a surface electroencephalogram (EEG). The functional connectivity parameters may be used to train a classifier (e.g., a machine learning model or network) to predict a treatment outcome. In one embodiment, the classifier may be trained to predict whether a patient in refractory epilepticus will successfully wean from IV anesthesia. In another embodiment, the classifier may be trained to predict clinical response to AED therapy for treatment of disorders of consciousness (e.g., seizures, IIC activity) after acute brain injury.

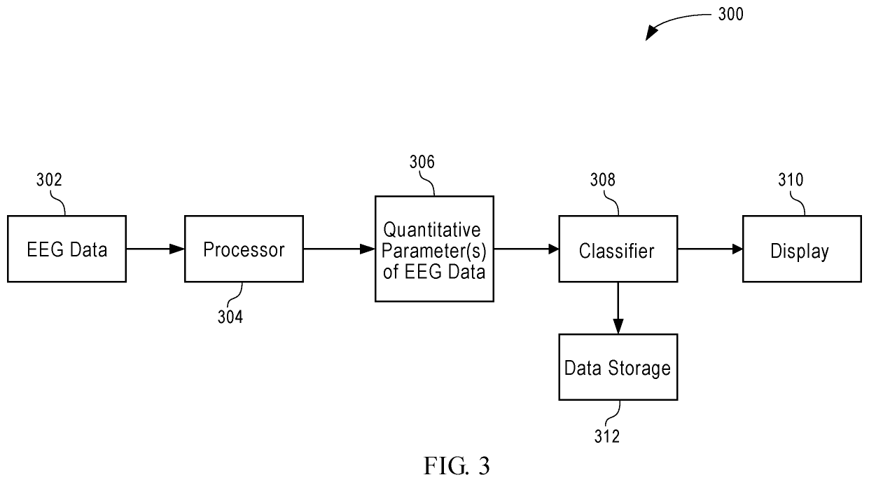

FIG. 3 is a schematic block diagram of a system for determining a treatment outcome for neurological disorders based on functional connectivity parameters in accordance with an embodiment. System 100 includes a processor (or controller) 304, a classifier 308, a display 310 and data storage (or memory) 312. EEG data 302 for a subject may be input into the processor 304. The EEG data may be acquired from a subject using a monitoring system such as, for example, the monitoring systems 100, 216 described above with respect to FIGS. 1A-2. In an embodiment, the EEG data 302 is continuous electroencephalography (cEEG) data. In one embodiment, the EEG data 302 may be provided to the processor from a monitoring system in real-time. In another embodiment, the EEG data 302 may be retrieved from, for example, data storage (or memory) of the monitoring system (e.g., monitoring systems 100, 216 of FIGS. 1A-2) or other computer system. Processor 304 is configured to receive the EEG data 302 and to determine (or calculate) at least one quantitative parameter 306 of the EEG data. In an embodiment, the at least one quantitative parameter 306 is a functional connectivity parameter (or spatial domain EEG features), for example, a spatial-correlation based measure of functional connectivity. Functional connectivity parameters may include, but are not limited to, network density, clustering coefficient, characteristic path length, number of independent components, number of non-trivial components, size of the largest independent component, characteristic path length of the largest component, and clustering coefficient of the largest component. In another embodiment, the quantitative parameters 306 may also include spatial components of the EEG signal (or frequency domain EEG features) such as, for example, relative alpha power, relative theta power, relative delta power and alpha/delta ratio. Known methods may be used to calculate the quantitative parameters 306 of the EEG data. For example, a spatial-correlation-based analysis quantifying the correlation structure of cortical activity or frequency-based analysis quantifying the power within different spectral components of the EEG data. Processor 304 may be a processor in a monitoring system (e.g., monitoring systems 100, 216 of FIGS. 1A-2) or may be part of a separate computer system.

One or more quantitative parameters 306 are input into the classifier 308. The classifier 308 may be, for example, a machine learning system or model. In an embodiment, the classifier 308 is a supervised learning model or network such as, for example, a support vector machine (SVM) or a k-nearest neighbor (KNN) clustering model. Classifier 308 is configured to determine (or predict or generate) a treatment outcome based on the one or more quantitative parameters 306 of the EEG data 302. Classifier 308 may be trained using known methods for training a machine learning system or model. Classifier may be incorporated into a monitoring system (e.g., monitoring systems 100, 216 of FIGS. 1A-2) or may be incorporated into separate computer system. In an embodiment, the treatment outcome may be provided to and displayed on a display 310. For example, the display 310 may be part of a monitoring system (e.g., monitoring systems 100, 216 of FIGS. 1A-2) or may be separate from a monitoring system. The treatment outcome may also be stored in data storage (or memory) 312 of, for example, a monitoring system (e.g., monitoring systems 100, 216 of FIGS. 1A-2) or other computer system. As mentioned above, in one embodiment the classifier 308 may be trained to predict whether a patient in refractory epilepticus will successfully or unsuccessfully wean from IV anesthesia. In another embodiment, the classifier 308 may be trained to predict a clinical response to AED therapy for treatment of disorders of consciousness (e.g., seizures, IIC activity) after acute brain injury.

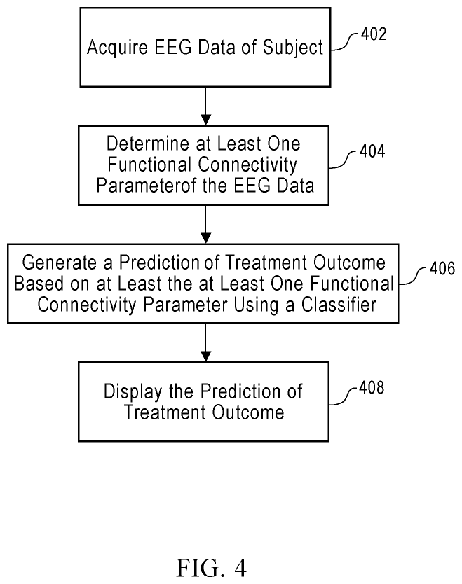

FIG. 4 illustrates a method for determining a treatment outcome for neurological disorders based on functional connectivity parameters in accordance with an embodiment. At block 402, EEG data of a subject is acquired. In an embodiment, the EEG data may be acquired from a subject using a monitoring system such as, for example, the monitoring systems 100, 216 described above with respect to FIGS. 1A-2. In an embodiment, the EEG data is continuous electroencephalography (cEEG) data. In one embodiment, the EEG data may be acquired in real-time. In another embodiment, the EEG data may be retrieved from, for example, data storage (or memory) of the monitoring system (e.g., monitoring systems 100, 216 of FIGS. 1A-2) or other computer system. At block 404, at least one functional connectivity parameter of the EEG data is determined or calculated using, for example, a processor (e.g., processor 304 in FIG. 3). As mentioned above, the at least one functional connectivity parameter (or spatial domain EEG feature), may be, for example, a spatial-correlation based measure of functional connectivity. Functional connectivity parameters may include, but are not limited to, network density, clustering coefficient, characteristic path length, number of independent components, number of non-trivial components, size of the largest independent component, characteristic path length of the largest component, and clustering coefficient of the largest component. Known methods may be used to calculate the functional connectivity parameters. In another embodiment, additional quantitative parameters may also be determined including spatial components of the EEG signal (or frequency-based parameters) such as, for example, relative alpha power, relative theta power, relative delta power and alpha/delta ratio. Known methods may be used to calculate the spatial components of the EEG data.

At block 406, a classifier is used to generate a prediction of a treatment outcome based on at least the at least one functional connectivity parameter. The classifier may be, for example, a machine learning system or model. In an embodiment, the classifier is a supervised learning model or network such as, for example, a support vector machine (SVM) or a k-nearest neighbor (KNN) model. At block 408, the prediction of the treatment outcome may be displayed on a display, for example, a display incorporated in a monitoring system (e.g., monitoring systems 100, 216 of FIGS. 1A-2) or a display separate from a monitoring system. In another embodiment, the prediction of treatment outcome may also be stored in data storage (or memory), for example, data storage of a monitoring system (e.g., monitoring systems 100, 216 of FIGS. 1A-2) or other computer system. In one embodiment, the prediction of a treatment outcome is determining whether a patient in refractory epilepticus will successfully or unsuccessfully wean from IV anesthesia. In another embodiment, the prediction of a treatment outcome is determining a clinical response to AED therapy for treatment of disorders of consciousness (e.g., seizures, IIC activity) after acute brain injury.

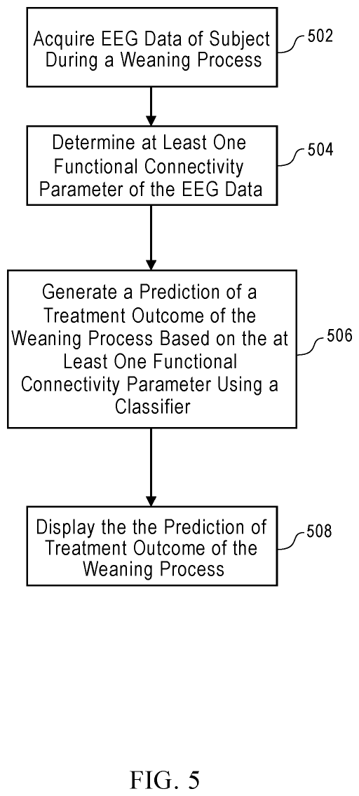

As mentioned above, in one embodiment a classifier may be trained to determine or predict a treatment outcome for a weaning process from anesthesia. FIG. 5 illustrates a method for determining an outcome of weaning of anesthesia in status epilepticus based on functional connectivity parameters in accordance with an embodiment. As used herein, a successful anesthetic wean is defined as the discontinuation of intravenous anesthesia without developing recurrent status epilepticus, and an unsuccessful wean (or wean failure) as either recurrent status epilepticus or the resumption of anesthesia for the purpose of treating an EEG pattern concerning for incipient status epilepticus.

At block 502, EEG data of a subject in refractory status epilepticus is acquired during a weaning process from an anesthetic agent (e.g., IV-TLA). In an embodiment, the EEG data may be acquired from a subject using a monitoring system such as, for example, the monitoring systems 100, 216 described above with respect to FIGS. 1A-2. In an embodiment, the EEG data is continuous electroencephalography (cEEG) data. In one embodiment, the EEG data may be acquired in real-time. In another embodiment, the EEG data may be retrieved from, for example, data storage (or memory) of the monitoring system (e.g., monitoring systems 100, 216 of FIGS. 1A-2) or other computer system. At block 504, at least one functional connectivity parameter of the EEG data is determined using, for example, a processor (e.g., processor 304 in FIG. 3). Known methods may be used to determine or calculate the functional connectivity parameters. The functional connectivity parameters may include one or more of network density, clustering coefficient, characteristic path length, number of independent components, number of non-trivial components, size of the largest independent component, characteristic path length of the largest component, and clustering coefficient of the largest component. In an embodiment, the functional connectivity parameters may also include on or more of spike frequency count, hemispheric asymmetry, focal rhythmicity, autocorrelation, and entropy.

At block 506, a classifier (e.g., classifier 308 in FIG. 3) is used to generate a prediction of a treatment outcome of the weaning process based on the at least one functional connectivity parameter. As discussed further below, distinct signatures in the spatial networks of functional connectivity emerge during successful anesthetic liberation in status epilepticus but these distinct signatures in the spatial networks of functional connectivity are absent in patients with anesthetic wean failure. Accordingly, successful and unsuccessful anesthetic weans can be predicted based on the one or more functional connectivity parameters of the EEG data for a subject in RSE. In an embodiment, the prediction of treatment outcome of the weaning process classifies the wean for a particular subject in refractory status epilepticus as either a wean that will be a successful or a wean that will be unsuccessful. The classifier may be, for example, a machine learning system or model such as a supervised learning model or network. In an embodiment, the classifier may be a support vector machine (SVM) and the output of the classifier is a time-varying score based on the distance from the discrimination boundary in the SVM. For example, a positive score may be used to indicate a prediction of a successful weaning based on the current EEG connectivity structure for the subject, a negative score may be used to indicate a prediction of a wean failure, and the magnitude of the score may provide a marker of confidence in the prediction. In one embodiment, the classifier is trained as a late-epoch prediction model. In another embodiment, the classifier is trained as an early-epoch time-varying prediction model.

At block 508, the prediction of the treatment outcome may be displayed on a display, for example, a display incorporated in a monitoring system (e.g., monitoring systems 100, 216 of FIGS. 1A-2) or a display separate from a monitoring system. In another embodiment, the prediction of treatment outcome may also be stored in data storage (or memory), for example, data storage of a monitoring system (e.g., monitoring systems 100, 216 of FIGS. 1A-2) or other computer system. The classifier may be used to help guide clinicians in the neuro ICU with, for example, identifying when patients are safe to wean from IV anesthesia. The classifier may be used to provide tailored, patient-specific treatment which can result in shorter courses of IV anesthesia, shorter ICU courses, and improved patient outcomes (e.g., faster and more successful anesthetic liberation after RSE). In an embodiment, the classifier may be used to analyze EEG data for a patient in RSE in real-time in an ICU, for example, as part of a bedside analytic tool.

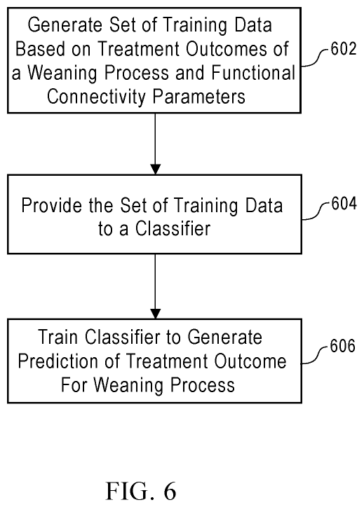

The classifier may be trained to predict wean outcomes using a set of training data that includes functional connectivity parameters. FIG. 6 illustrates a method for training a classifier for determining an outcome of weaning of anesthesia in status epilepticus based on functional connectivity parameters in accordance with an embodiment. At block 602, a set of training data is generated based on treatment outcomes of a weaning process and functional connectivity parameters. The set of training data may be formed using existing EEG data (e.g., cEEG data) recorded from patients undergoing weaning of an anesthetic agent (e.g., IV-TLA). The existing EEG data may be retrieved from, for example, a database. In one embodiment, EEG data associated with a consecutive series of patients diagnosed with RSE over a predetermined time period (e.g., a number of years) who were treated with at least one intravenous anesthetic agent may be used to form the set of training data. A set of quantitative functional connectivity parameters can be calculated for each individual wean based on the associated EEG data. In addition, each individual wean can also be classified as successful or unsuccessful based on the clinical outcome. For example, the definitions of an attempted wean as well as the definitions of wean success and wean failure may be pre-specified. In an embodiment, an attempted wean was defined as the cessation of continuous infusion of IV anesthetics. Medical records associated with each attempted wean may then be used to confirm that each attempted wean was intended for the purpose of liberating the patient from IV-TLA therapy, as opposed to a temporary pause for a neurologic exam. Once confirmed, each attempted wean may then be classified as either successful or unsuccessful. As mentioned above, a successful wean may be defined as the cessation of IV anesthetics without the development of recurrent status epilepticus for a predetermined period of time, for example, for at least 48 hours and an unsuccessful wean may be defined as either recurrent status epilepticus or the resumption of IV anesthetics due to clinical or electrographic concern for worsening clinical features on cEEG (e.g. an increase in IIC burden or frequency). In one embodiment, the resumption of IV anesthetics specifically for the annotated purpose of patient comfort during intubation is not considered a wean failure. In such cases the wean could either be a success if the patient remained free from recurrent status during a predetermined time period (e.g., for 48 hours) or a failure if recurrent status occurred or anesthetic therapy was subsequently escalated for the purpose of treating a concerning clinical syndrome or IIC EEG pattern. In an embodiment, the set of training data may be formed using EEG data for each individual wean from a predetermined time period, for example, a predetermined amount of time before the cessation of anesthesia or earlier time series.

At block 604, the training data is provided to a classifier which may be for example, a supervised learning model or network such as a support vector machine (SVM). At block 606, the classifier is trained to generate a prediction of treatment outcomes for a weaning process using the generated set of training data. As discussed above, each individual wean in the set of training data has a set of calculated functional connectivity parameters and a classification as either successful or unsuccessful. In one embodiment, the classifier may be trained as a late-epoch prediction model using, for example, a set of training data calculated based on the EEG data during a time period (e.g., 30 minutes) preceding the end of each attempted wean. In an example where the classifier is an SVM, the set of training data for a particular epoch may be used to train the SVM using a linear kernel to distinguish between successful and unsuccessful weans. In another embodiment, the classifier may be trained as an early-epoch time-varying prediction model by, for example, applying the late-epoch classifier to all earlier epochs from each individual wean, such that the resulting model was applied to the full cEEG record to produce a time-varying “score” that may be used to predict wean success.

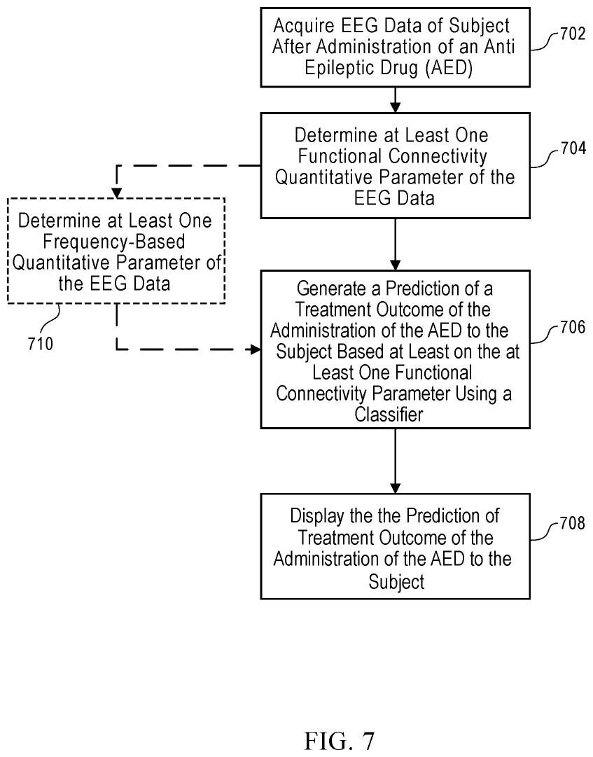

As mentioned above, in another embodiment the classifier may be trained to determine or predict a treatment outcome of the application (or administration) of an AED to a subject. FIG. 7 illustrates a method for determining an outcome of treatment of a subject after acute brain injury with an antiepileptic drug based on functional connectivity parameters in accordance with an embodiment. At block 702, EEG data for a subject is acquired after administration of an anti-epileptic drug (AED) to the subject. In an embodiment, the EEG data may be acquired from a subject using a monitoring system such as, for example, the monitoring systems 100, 216 described above with respect to FIGS. 1A-2. In an embodiment, the EEG data is continuous electroencephalography (cEEG) data. In one embodiment, the EEG data may be acquired in real-time. In another embodiment, the EEG data may be retrieved from, for example, data storage (or memory) of the monitoring system (e.g., monitoring systems 100, 216 of FIGS. 1A-2) or other computer system. At block 704, at least one functional connectivity quantitative parameter of the EEG data is determined using, for example, a processor (e.g., processor 304 in FIG. 3). Known methods may be used to determine or calculate the functional connectivity parameters. The functional connectivity parameters may include, for example, network density, clustering coefficient, characteristic path length, number of independent components, number of non-trivial components, size of the largest independent component, characteristic path length of the largest component, and clustering coefficient of the largest component. In an embodiment, the functional connectivity parameters may also include, for example, spike count and frequency, entropy and hemispheric specific measures. In an embodiment, at least one frequency-based quantitative parameter of the EEG data is also determined at block 710. Known methods may be used to determine or calculate the frequency-based parameters. The frequency-based parameters may include, for example, relative alpha power, relative theta power, relative delta power and alpha/delta ratio.

At block 706, a classifier is used to generate a prediction of treatment outcome of the administration of the AED to the subject based at least on the at least one functional connectivity parameter. In an embodiment, the classifier may also generate the prediction of treatment outcome based at least one frequency-based parameter of the EEG data in addition to the functional connectivity parameters. Empirically intensifying AED treatment for disorders of consciousness after ABI has diminishing benefit after the initial administration of an AED. As discussed further below, early response to treatment may be used to predict clinical response following AED treatment. By using quantitative EEG biomarkers (e.g., the functional connectivity parameters and frequency-based parameters) of early treatment response to an AED, the classifier may robustly predict clinical response following AED treatment. In an embodiment, the output of the classifier is prediction of a change in a Glasgow Coma Scale (GCS) score over a predetermined period of time, for example, 24 hours after treatment of the subject with an AED For example, the classifier may classify segments of the EEG data for a specific patient input to the classifier into anticipated GCS score trajectory group. In an example, the GCS trajectory groups may include (1) improve (an improvement in GCS score), (2) decline (a decline in GCS score), and (3) stable (no change in GCS score). In an embodiment, the sign of the change in GCS score after the predetermined time after administration of the AED may be used as the outcome label. The classifier may be, for example, a machine learning system or model such as a supervised learning model or network. The classifier may be, for example, a machine learning system or model such as a supervised learning model or network. In an embodiment, the classifier may be a K-nearest neighbor (KNN) clustering algorithm.

At block 708, the prediction of the treatment outcome may be displayed on a display, for example, a display incorporated in a monitoring system (e.g., monitoring systems 100, 216 of FIGS. 1A-2) or a display separate from a monitoring system. In another embodiment, the prediction of treatment outcome may also be stored in data storage (or memory), for example, data storage of a monitoring system (e.g., monitoring systems 100, 216 of FIGS. 1A-2) or other computer system. The classifier may be used to identify patients most likely to benefit from AED therapy, so that such patients may be treated promptly and spared the sequalae of potentially injurious electrographic brain activity, while simultaneously aiding clinicians in appropriately deferring AED therapy on patients likely only to experience negative side effects of AED therapy, most notably sedation, arrythmia, and hepatoxicity. Accordingly, the classifier may be used to improve the care of patients admitted for the management of acute brain injury. In an embodiment, the classifier may be used to analyze EEG data for a patient with ABI in real-time, for example, as part of a bedside analytic tool.

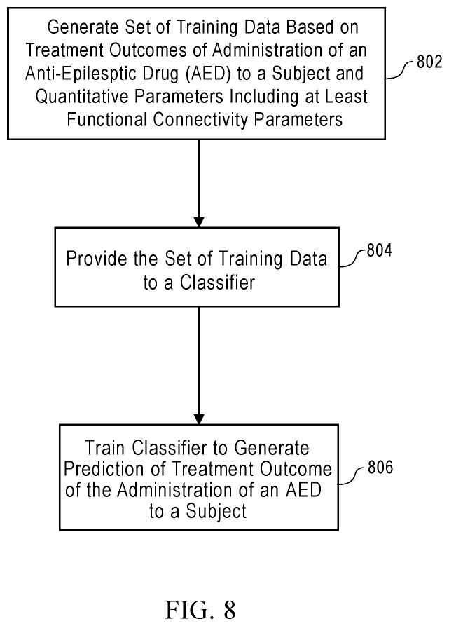

The classifier may be trained to predict a treatment outcome of the administration of an AED to a subject using a set of training data that includes quantitative parameter, for example, functional connectivity parameters and frequency-based parameters. FIG. 8 illustrates a method for training a classifier for determining an outcome of treatment of a subject after acute brain injury with an antiepileptic drug based on functional connectivity parameters in accordance with an embodiment. At block 802, a set of training data is generated based on quantitative parameters including functional connectivity parameters and frequency-based parameters and a change (or trajectory) of a GCS score over a period of time after administration of an AED. The set of training data may be formed using clinical data and existing EEG data (e.g., cEEG data) recorded from patients including EEG data acquired after the administration of an AED (a medication administration event or event). The existing EEG data and clinical data may be retrieved from, for example, a database. In one embodiment, the EEG data and clinical data is associated with patients undergoing continuous electroencephalography (cEEG) during admission for ABI. In an embodiment, EEG and clinical data for patients with diagnoses of subarachnoid hemorrhage, traumatic brain injury (including traumatic intracranial hemorrhage), and spontaneous intracerebral hemorrhage may be included in the training data. The clinical data used to generate the set of training data may include, for example, regularly sampled GCS scores (e.g., over the duration of the ICU admission) and medication dose administration records. Data such as the duration of the duration of the ICU admission and the time, dose, and route of administration of all antiepileptic medications may be retrieved from the medication dose administration records. In an embodiment, each patients EEG may also be classified by the presence or absence of ictal interictal continuum (IIC) activity. Based on the sign of a change in the GCS score over a predetermined period of time (e.g., 24 hours), each event (i.e., the administration of AED) for each patient is assigned to one of three categories: decline, stable, or improve. In addition, a set of quantitative parameters including one or more functional connectivity parameters and frequency-based parameters can be calculated for each individual EEG associated with a patient. In an embodiment, the quantitative parameters may be calculated over a predetermined period after the administration of the AED In another embodiment, the quantitative parameters may also be calculated for a period of time before the administration of the AED

At block 804, the training data is provided to a classifier which may be for example, a supervised learning model or network such as a K-nearest neighbor (KNN) clustering algorithm. At block 806, the classifier is trained to generate a prediction of treatment outcomes of the administration of an AED to a subject using the generated set of training data. Known methods may be used to train the classifier. As discussed above, the data for each patient in the set of training data has a set of calculated quantitative parameters and a classification of the GCS trajectory of decline, stable or improve. In one embodiment, the classifier is trained to classify segments of the EEG data for a specific patient input to the classifier into anticipated GCS score trajectory group. In an example, the GCS trajectory groups may include (1) improve (an improvement in GCS score), (2) decline (a decline in GCS score), and (3) stable (no change in GCS score.

The following example sets forth, in detail, ways in which the present disclosure was evaluated and ways in which the present disclosure may be used or implemented, and will enable one of ordinary skill in the art to more readily understand the principles thereof. The following examples are presented by way of illustration and are not meant to be limiting in any way.

### Example 1

The training and use of a classifier configured to determine or predict, based on a surface EEG recording, whether or not a patient will successfully wean from IV anesthesia was evaluated. As mentioned above, in this embodiment the classifier is trained using functional connectivity parameters for EEGs in a set of training data. By analyzing changes in functional connectivity parameters of the network activity captured on surface EEG, significant differences in network structure may be identified between patients that do or do not successfully wean from IV anesthetics. In this example, a successful anesthetic wean was pre-specified as the discontinuation of intravenous anesthesia without developing recurrent status epilepticus, and a wean failure was pre-specified as either recurrent status epilepticus or the resumption of anesthesia for the purpose of treating an EEG pattern concerning for incipient status epilepticus. Two types of features were evaluated as predictors of successful weaning: (a) spectral components of the EEG signal, and (b) spatial-correlation-based measures of functional connectivity. Spectral components of the EEG revealed no significant differences between successful and unsuccessful weans. Analysis of functional connectivity measures revealed that successful anesthetic weans were characterized by the emergence of larger, more densely connected, and more highly clustered spatial functional networks, yielding 75.5% (95% confidence interval: 73.1%-77.8%) testing accuracy in a bootstrap analysis using a hold-out sample of 20% of data for testing and 74.6% (95% confidence interval 73.2%-75.9%) testing accuracy in a secondary external validation cohort, with an area under the curve of 83.3%. The results of these analyses were used to train a classifier to predict wean outcome.

To determine whether quantitative features of continuous EEG (cEEG) could be used to guide anesthetic treatment for RSE, a series of analyses were performed on cEEG data recorded from patients undergoing weaning of IV-TLA. Two modes of analysis were performed: (a) a frequency-based analysis quantifying the power within different spectral components of the cEEG, and (b) a spatial-correlation-based analysis quantifying the correlation structure of cortical activity. Individual anesthetic weans in a set of training data may be classified as either successful or unsuccessful based on clinical outcome, and both frequency-domain and spatial-domain cEEG features were compared. In this example, it is shown that successful and unsuccessful anesthetic weans can be reliably distinguished by a set of quantitative cEEG features that, therefore, could be used to predict the subsequent outcome of an individual anesthetic wean.

In this example, from a single-center prospectively collected cohort of patients' continuous cEEG data, a consecutive series of patients diagnosed with RSE over a number of years were identified who were treated with at least one IV-TLA. For the training data used in this example, patients diagnosed with status epilepticus in the setting of cardiac arrest were excluded and patients with all other underlying diagnoses were included.

In this example, an index test was used that included several pre-specified frequency-domain and spatial correlation measures of functional connectivity on cEEG. There is an association between changes in relative alpha power (8-13 Hz) and the alpha-delta ratio and neurologic decline in other forms of acute neurologic injury including subarachnoid hemorrhage, ischemic stroke, and post-anoxic coma. Accordingly, power in the alpha frequency spectrum (8-13 Hz) and the ratio of alpha to delta power were specified as the main frequency-domain cEEG measures from several frequency-specific measures including power in the theta (4-8 Hz) and delta (0.5-4 Hz) frequency spectra. Given that most network statistics are driven by network density, network density was pre-specified as the main index test of functional connectivity from a standard battery of spatial correlation measures of functional connectivity. In addition to network density, a standard series of graph-theoretical parameters describing the topology of the functional network were derived: the clustering coefficient, the characteristic path length, the number of independent components, the number of non-trivial components, the size of the largest independent component, the characteristic path length of the largest component, and the clustering coefficient of the largest component. The cEEG recordings used in this example were acquired utilizing a standard 10-20 electrode arrangement at either 256 or 512 Hz. For the analysis performed, recordings were low-pass filtered at 125 Hz using a third-order Butterworth filter, notch filtered at 60 Hz and 120 Hz to reduce line noise, and referenced to the average.

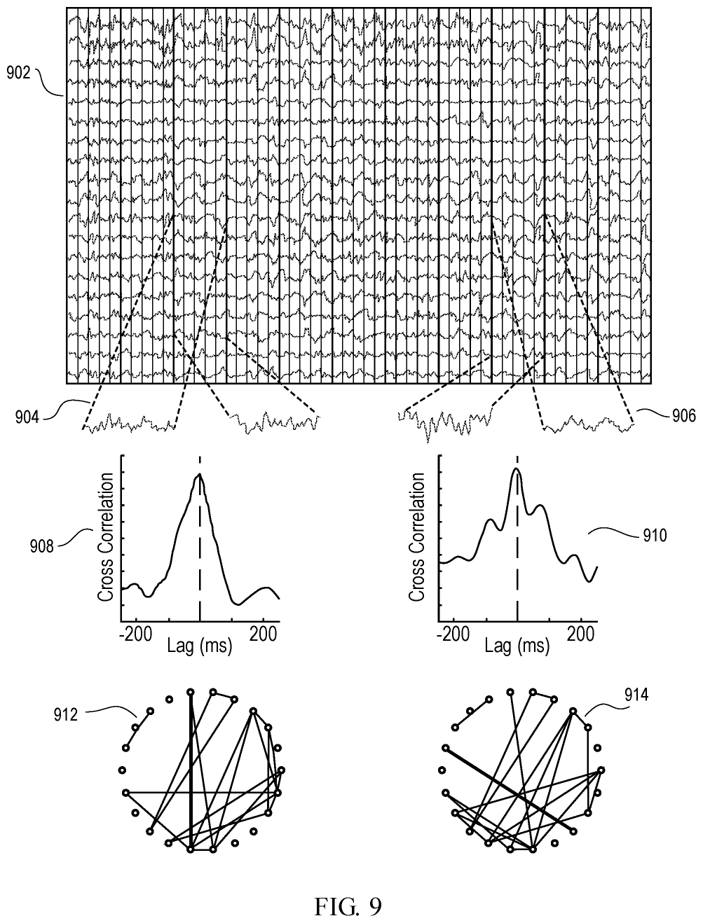

Each of the index test measures were calculated based on trends of frequency and time-varying maps of the functional connectivity. FIG. 9 illustrates a method for calculating functional connectivity from cEEG signals in accordance with an embodiment. In this example, the functional connectivity parameters were calculated in 1000-ms intervals with 500-ms overlap between intervals. In FIG. 9, an example of 11 seconds of cEEG data 902 from a patient undergoing an anesthetic wean is shown as well as two pairs 904, 906 of 1-second long EEG tracings. Power within the alpha, theta, and delta bands was normalized relative to the overall power (0.5-20 Hz) in the EEG signal. For the calculation of functional connectivity, within each 1000-ms segment, the signal at each electrode was z-normalized to zero mean and unit variance over the 1000-ms interval. Normalization was performed on the signal from each electrode independently for each patient. A cross-correlogram of the EEG activity was then calculated between all pairs of electrodes using a +/−250-ms window of overlap. For example, in FIG. 9, the cross correlations 908, 910 between pairs 904, 906, respectively, are shown. The peak in the cross-correlogram between each pair of electrodes was identified, and statistically significant peaks in the cross-correlation defined a connection. For each 1000-ms epoch of data, a network of nodes (the EEG electrodes) and edges (the statistically-significant cross-correlations defining the connections between them) was calculated. In FIG. 9, examples of the functional connectivity maps 912, 914 are shown, with circles (the “nodes” of the functional connectivity maps) representing each EEG lead and lines (the “edges” of the functional connectivity maps) representing connections defined by a statistically significant peak in the cross-correlogram between the signals recorded from each lead. From each functional connectivity network, the graph theoretical measures described above were calculated. In this example, the analysis was not restricted to data without artifact.

The index test metrics above (alpha power, ratio of alpha to delta power, and network density) were then tested for differences between the reference standard of the successful or failed wean of IV-TLA infusions. As mentioned above, both the definition of an attempted wean as well as the definitions of wean success and wean failure were pre-specified. Medication administration records with time-stamped records of medications, route, concentration, and rate were reviewed. An attempted wean was defined as the cessation of continuous infusion of IV anesthetics. Medical records were then reviewed to confirm that each attempted wean was intended for the purpose of liberating the patient from IV-TLA therapy, as opposed to a temporary pause for a neurologic exam. Once confirmed, weans were then classified as either successful or unsuccessful. A successful wean was defined as the cessation of IV anesthetics without the development of recurrent status epilepticus for at least 48 hours. An unsuccessful wean was defined as either recurrent status epilepticus or the resumption of IV anesthetics due to clinical or electrographic concern for worsening clinical features on cEEG (e.g. an increase in IIC burden or frequency). However, the resumption of IV anesthetics specifically for the annotated purpose of patient comfort during intubation was not considered a wean failure. In such cases the wean could either be a success if the patient remained free from recurrent status for 48 hours or a failure if recurrent status occurred or anesthetic therapy was subsequently escalated for the purpose of treating a concerning clinical syndrome or IIC EEG pattern. Recurrent status epilepticus was defined and includes recurrent seizures with evolution and without intervening return to baseline, epileptiform activity with a clinical correlate, or, alternatively, periodic discharges with frequency of 2.5 Hz or greater.

In this example, an outcome was determining the differences in spectral power and functional connectivity metrics between successful and unsuccessful weans. Initially, to compare successful and unsuccessful weans, a 30-minute period culminating at the time of anesthesia cessation was identified. The index tests were then averaged over this epoch for each wean. The value of each of these parameters was then compared between successful and failed weans, allowing for no indeterminate test results. To determine whether cEEG preceding these 30-minute segments was also predictive at earlier time periods during the wean, the same comparison was performed over the earlier time-series for each parameter, specifying time=0 as the time of cessation of IV anesthetic. The successful and unsuccessful weans were again compared.

To measure differences in the index test between wean success and wean failure reference standard outcomes, in this example unpaired two-tailed Student's t-test were performed after confirming that parameter sets were adequately normally distributed by a Kolmogorov-Smirnov test of normality. Where direct comparisons of multiple simultaneous parameters were performed, a Holm-Bonferroni procedure was used to correct for multiple comparisons. A corrected p-value of <0.05 was considered as statistically significant. In this example, it was also sought to determine whether the differences described above could be used to train a classifier to predict whether a given anesthetic wean was likely to be successful based on cEEG characteristics. To do this, a support vector machine (e.g., a classifier) was trained using the quantitative parameters calculated as described above. As described below, two SVM prediction models (e.g., classifiers) were evaluated, namely a late-epoch prediction model and an early-epoch prediction model.

A late-epoch prediction model may be calculated based on the results of the primary statistical measures during the 30 minutes preceding the end of each attempted wean. For this 30-minute epoch, the calculated quantitative metrics were used to train a support vector machine (SVM) model using a linear kernel to distinguish between successful and unsuccessful weans. To assess the accuracy of the classifier, a 100-fold cross validation process was performed. In each iteration, data from 80% of the patients were used to train the classifier and data from 20% of the patients were withheld for testing. The classifier was then used to predict the outcome on the withheld 20% of data, and the accuracy of the classifier was calculated by comparing the predicted outcome to the known outcome in each withheld wean. As a control, a second classifier was trained on the same 80% of the data after randomly shuffling the reference standard outcome measures. This process was repeated 100 times and an average accuracy was calculated for both the true and control classifier. The predictive accuracy of the control model was compared to the model trained on true outcome data.

As an additional test of robustness, the predictive model trained on the primary data set was then used to predict the outcome of anesthetic weans on an additional validation cohort of 15 anesthetic weans. In each of 100-iterations, data from 80% of the patients from the primary data set were used to train both the classifier and control models. These two models were then used to predict the outcome of 80% of the weans from the validation cohort and again the predictive accuracy of the classifier model and control models was compared.

An early-epoch time-varying prediction model was then developed by applying the classifier to all earlier epochs from each wean, such that the resulting model was applied to the full cEEG record to produce a time-varying “score” that could be used to predict wean success. The model was again trained using data from the 30-minute segments prior to IV-TLA cessation but was then tested at all preceding time points. As in the late-epoch model, on each iteration of a 100-fold cross-validation, weans from 80% of the patients were randomly selected as training data. The trained model was then applied to the full time series data from the withheld 20% of the data, and the prediction accuracy was calculated as a function of time. The model was again compared to a control model trained on randomly shuffled outcome labels.

For each of the late-epoch predictive model and the early-epoch predictive model in this example, a time-varying “score” was output based on the distance from the discrimination boundary in the SVM. In an embodiment, a positive score predicts successful weaning based on the current EEG connectivity structure, a negative score predicts wean failure, and the magnitude of the score provides a marker of confidence in the prediction. Because some of the patients underwent multiple weans, it is possible that the magnitude of some of the differences that were observed were due to within-subject oversampling. To control for this possibility, the initial analysis comparing the functional connectivity parameters and spectral power between the two groups were repeated using only a single wean from each patient.

To create a training data set for this example, forty-seven consecutive anesthetic weans (23 successes, 24 failures) were identified from a single-center cohort of patients admitted with refractory status epilepticus from 2016-2019. A total of 47 anesthetic weans (23 successful, 24 unsuccessful) in 34 patients met criteria for inclusion and were included in the primary cohort. The age of the patients ranged from 20-85 years old (median age 65); 20 of the 34 patients were female. The causes of status epilepticus in this cohort included autoimmune/paraneoplastic and infectious encephalitides, stroke, subdural hematoma, subarachnoid hemorrhage, metabolic derangement, traumatic brain injury, primary epilepsy syndromes, and primary and metastatic brain tumors. Eight of the 34 patients underwent more than 1 anesthetic wean; six patients underwent two weans, one patient underwent three weans, and one patient underwent six weans. Following conclusion of the primary analysis, a secondary validation cohort of an additional 15 anesthetic weans in 13 patients was assembled using the same inclusion criteria.

In the data used for this example, the most commonly used IV-TLA was propofol (in 40 weans) and the second most frequently used IV-TLA was midazolam (in 11 weans).

Pentobarbital was used in five weans and ketamine was used in four weans. Eleven weans involved the use of more than one IV-TLA. Of the 40 anesthetic weans that included propofol, 20 (50%) were successes and 20 (50%) were failure. Of the 7 anesthetic weans that did not include propofol as one of the IV-TLA, 3 (43%) were successes and 4 (57%) were failures. There was no difference in outcome between weans that did and did not include propofol (p=1.0, two-tailed Fisher's exact test). The duration of IV anesthetic weans (measured as the time from peak dose to cessation of infusion) ranged from 1 minute (in cases in which anesthetics were discontinued abruptly) to 69.8 hours. The median duration for successful weans was 3.7 hours and for unsuccessful weans was 3.0 hours. There was no significant relationship between wean duration and likelihood of success (U=264, p=0.80, two-tailed Mann-Whitney U test). EEG demonstrated a burst suppression pattern prior to weaning in 18 of the anesthetic weans, of which 7 (39%) were successful. Of those anesthetic weans that did not follow a period of burst suppression, 16/29 (55%) were successful. There was no difference in outcome between weans that did or did not follow a period of burst suppression (p=0.37, two-tailed Fisher's exact test). The conventional AEDs used varied considerably and the two most commonly used were levetiracetam (used in 43/47 weans) and phenytoin (used in 32/47 weans).

In this example, twelve parameters were determined and evaluated. Examples of the parameters include four frequency-based measures (e.g., relative alpha power, relative theta power, relative delta power, and alpha/delta ratio) and eight spatial-correlation-based functional connectivity measures (e.g., network density, clustering coefficient, characteristic path length, number of independent components, number of non-trivial components, size of the largest independent component, characteristic path length of the largest component, and clustering coefficient of the largest component). Several observations may be made based on time-series trends for the parameters over the course of a wean. In most cases, the cessation of intravenous anesthesia led to a rise in the alpha/delta ratio, regardless of wean outcome. In many of the successful weans, an increase in network density, a decrease in the number of independent components, an increase in the clustering coefficient, and an increase in the size of the largest component were apparent. Overall, the networks appeared become more spatially connected in these cases. These same changes appeared largely absent from the unsuccessful weans. In both the successful and unsuccessful cases, the alpha/delta ratio rises as IV anesthesia is discontinued. In the successful but not the unsuccessful case, as IV anesthesia is withdrawn, there is a gradual rise in network density, clustering coefficient, characteristic path length, and size of the largest component and fall in the number of independent components as network connectivity rises.

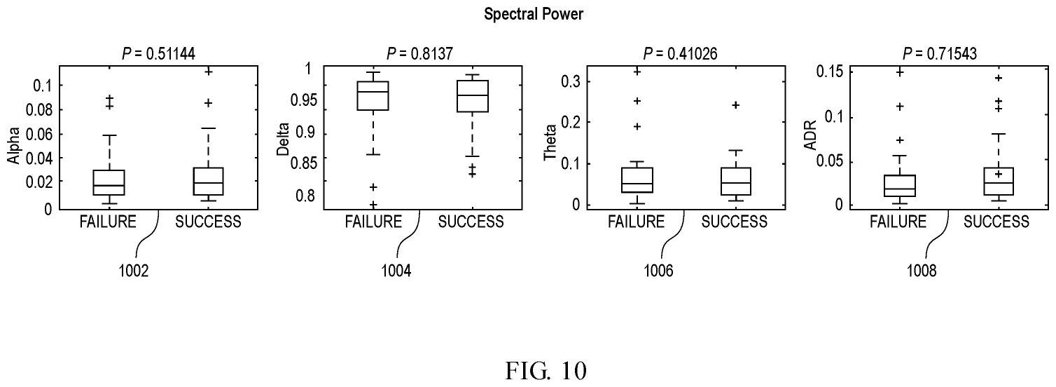

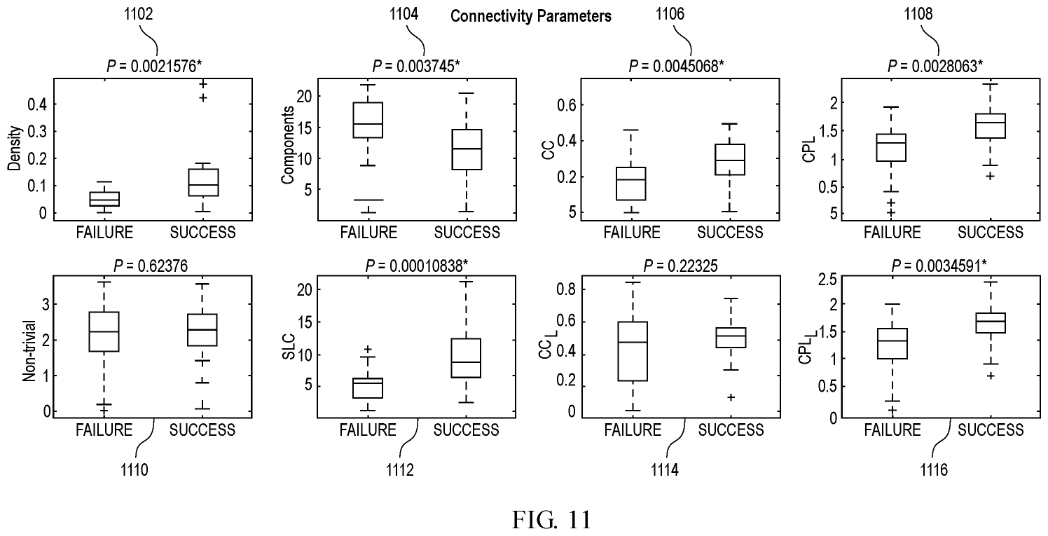

Group differences in cEEG index test measures between the wean success and wean failure reference standard outcomes during the 30-minute period prior to IV-TLA cessation are shown in FIGS. 10 and 11. FIG. 10 shows graphs illustrating a comparison of frequency-based parameters (or metrics) between successful and unsuccessful anesthesia weans in accordance with an embodiment. Between the successful and unsuccessful weans, there was no difference in the main frequency-specific outcomes, specifically relative alpha power 1002 (t=0.6619₄₅, p=0.51144, two-tailed Student's t-test) or the ratio of alpha to delta power 1008 (t=0.3669₄₅, p=0.71543; two-tailed Student's t-test). Additionally, there was no difference in relative delta 1004 (t=0.2370₄₅, p=0.8137, two-tailed Student's t-test) or theta power 1006 (t=0.8312₄₅, p=0.41026, two-tailed Student's t-test). Given the differential effects of IV anesthetics on EEG power within the beta range, the power within the betas (12.5-16 Hz) and beta2 (16-20 Hz) bands were also compared, and similarly no difference was found between successful and unsuccessful weans (beta: t=0.1950₄₅, p=0.8463, two-tailed Student's t-test; beta2: t=0.7625₄₅, p=0.4497, two-tailed Student's t-test). FIG. 11 shows graphs illustrating a comparison of functional connectivity parameters (or metrics) between successful and unsuccessful anesthesia weans in accordance with an embodiment. In contrast to the frequency-based parameters, there was a difference between the wean success and wean failure groups for the main functional connectivity index test, network density 1002 (t=3.254945, p=0.0021576, two-tailed Student's t-test). Overall, there were statistically significant differences for six of the eight parameters that characterize the functional connectivity of the network. Specifically, network density 1102, clustering coefficient 1106 (t=2.9903₄₅, p=0.0045068, two-tailed Student's t-test), characteristic path length 1108 (t=3.1617₄₅, p=0.0028063, two-tailed Student's t-test), size of the largest component 1112 (t=4.2437₄₅, p=0.00010838, two-tailed Student's t-test), and characteristic path length of the largest component 1116 (t=3.0867₄₅, p=0.0034591, two-tailed Student's t-test) were significantly higher during wean success, and the number of independent components was significantly lower (t=3.8467₄₅, p=0.00037₄₅, two-tailed Student's t-test) during wean success compared with wean failure. There was no significant difference in the number of non-trivial components 1104 (t=0.4939₄₅, p=0.62376, two-tailed Student's t-test) or the clustering coefficient of the largest component 1114 (t=1.2350₄₅, p=0.22325, two-tailed Student's t-test). As described above, in this example a 30-minute time segment was used for these comparisons; however, shorter and longer time periods of comparison yielded similar results.

Group differences in cEEG index test measures between the wean success and wean failure reference standard outcomes during earlier time points were analyzed. Due to the multi-hour duration of these cEEG segments, this data contains cEEG segments corrupted by ICU artifact, and some cEEG records were not available for all time periods. Nonetheless, these data from real-time clinical recordings confirm and extend the findings from the 30-minute analysis, demonstrating that up to 6 hours prior to discontinuation of anesthesia, that network density, clustering coefficient, and size of the largest component of the network are consistently greater and that the number of independent components is consistently lower in the wean success group than wean failure group. For some parameters, differences between the reference standard outcome groups are greatest earlier in the wean course.

As burst suppression may have an impact on network density and the subsequently evolution of other network features, it was also explored whether there was any difference in network parameters between those anesthetic weans that did or not follow a period of burst suppression. Similar to our finding that burst suppression did not significantly impact outcome, no significant difference in network features was seen between those anesthetic weans that did or not follow a period of burst suppression nor any significant difference in network density up to 24 hours prior to wean cessation.

In a post-hoc analysis for within-subject oversampling bias for this example, eight patients were identified that underwent multiple weans, and a total of 1,152 different possible combinations (6×3×2⁶=1,152) of weans using only a single wean from each patient. For each combination, the comparison of all twelve metrics was performed, and the p-values of the differences between the groups were calculated. In most cases the differences between the groups persisted. The histograms of the p-values were skewed leftward and p-values remained <0.05 in the majority cases for the 6 connectivity parameters in which a difference was noted in the full data set. In some instances of evaluating a single wean per patient, p-values within each outcome group were not small enough to be considered significant after both minimizing the data samples and subsequently correcting for multiple comparisons.

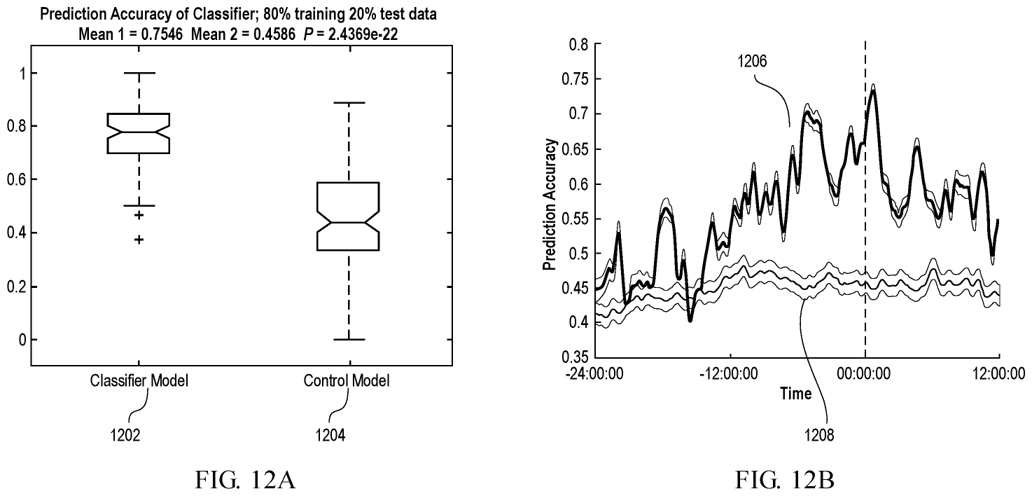

FIGS. 12A and 12B are graphs illustrating accuracy testing of a classifier in accordance with an embodiment. In this example, for each of 100 iterations the classifier was trained on data from a randomly selected 80% of patients, and then tested on the remaining 20% of the data. Data used for training and testing was calculated by averaging the quantitative metrics for each wean over the 30-minute period ending at the time of anesthesia cessation. The late-epoch prediction model 1202 developed in the training set could correctly predict the outcome of wean success with 76.4% training accuracy (95% CI: 75.7-77.0) and 75.5% testing accuracy (95% CI: 73.1%-77.8%) in the withheld data; the control model 1204 with randomly shuffled labels had 45.9% testing accuracy in the withheld data (χ2=94.5114₁, p=2.44×10⁻²², Kruskal-Wallis H test). When applied to the secondary external validation cohort, the prediction model had an accuracy of 74.6% (95% CI: 73.2-75.9); the control model with randomly shuffled labels had an accuracy of 47.3% (χ2=111.9262₁, p=3.71×10⁻²⁶, Kruskal-Wallis H test). The area under the curve of the receiver operating characteristic curve was 83.31% in the predictive model and 47.56% in the control model (χ2=122.0143₁, p=2.29×10⁻²⁸, Kruskal-Wallis H test). The early-epoch time-varying prediction model 1206 trained on the reference standard begins to diverge from that of the control model approximately 16 hours prior to wean cessation; by approximately 12 hours prior to wean cessation the prediction model outperforms the control model 1208 and accuracy improves over time.

As discussed above, this example demonstrates that quantitative cEEG measures of functional connectivity can be used to predict success in weaning from anesthesia (e.g., IV-TLA) in RSE. Connectivity parameters distinguishing between successful and unsuccessful anesthetic weans can be detected early in weaning and enable the use of a classifier tool that can predict wean success in a hold-out data set before the anesthetic weaning attempt is concluded. The pre-specified measure of network density and several associated measures of network connectivity showed good predictive accuracy during multiple time points during anesthetic weaning. However, no frequency domain measures discriminated wean success from wean failure in this cohort.

Successful weaning from IV anesthetics and recovery from RSE is preceded by cortical activity with significantly increased density, fewer independent components, higher clustering coefficients, and larger largest components. Though several of these measures are closely correlated and largely driven by network density, overall this pattern suggests that recovery from RSE is associated with more highly correlated cortical activity. Changes in functional network topology over the time course of individual seizures demonstrates that seizure termination is characterized by an increase in functional network density and size of the largest component of functional networks, both of which were similarly found to be characteristics of successful anesthetic weans in the present example. Neuronal network mechanisms of seizure termination may be shared by networks with resolved RSE. Moreover, it may be that network properties that support seizure propagation in the ictal state, when present in the interictal state, may predispose to recurrent seizures and the development of status epilepticus.

As discussed above, refractory status epilepticus yields significant morbidity attributable to the necessary but prolonged use of IV-TLA infusions. Successful weaning from IV-TLA in RSE is heralded by significant changes in the functional connectivity of cortical networks, and these differences can accurately predict anesthetic liberation at both early and late time points during an attempted anesthetic wean. These differences may be applied in the ICU to help minimize the duration of pharmacologically induced coma in patients with status epilepticus.

### Example 2

The training and use of a classifier configured to determine or predict a clinical response to AED therapy for treatment of disorders of consciousness (e.g., seizures, IIC activity) after acute brain injury was evaluated. As mentioned above, in this embodiment the classifier is trained using functional connectivity parameters and frequency-based parameters for EEGs in a set of training data. In this example, EEG and electronic medical records are analyzed and a functional connectivity and machine learning paradigm is employed to identify electrographic biomarkers predicting clinical response to AED therapy.

Patients undergoing continuous electroencephalography (cEEG) during admission for ABI were selected from a prospective big data repository of clinical data including regularly sampled Glasgow Coma Scale (GCS) scores and medication dose administration records. Frequency-specific spectral power (e.g., alpha 8-13 Hz, theta 4-7 Hz, and delta 0.5-4 Hz) and graph theoretical metrics of EEG functional connectivity were compared at time intervals before and after AED therapy. 204 patients met inclusion criteria, of whom 195 received at least one AED. In this example, initiation of a first AED was associated with a 0.54 point improvement in the GCS score after 24 hours and empiric escalation of AED regimen beyond a single agent was correlated with a decline in GCS score. A machine learning algorithm (e.g., a classifier) was trained on quantitative (qEEG) metrics derived from epochs of EEG data recorded after AED therapy, using as an outcome the change in GCS score 24 hours after treatment. When tested on qEEG data from a withheld subset of patients, this algorithm can accurately predict the subsequent trajectory of GCS score.

In this example, a series of quantitative analytic tools were employed to identify specific electrographic biomarkers predicting clinical response to AED therapy. From a prospectively gathered repository of clinical data of patients admitted to the intensive care unit, adult patients were selected that had undergone continuous electroencephalography (cEEG) during admission to the intensive care unit for ABI over a number of years. In this example, inclusion criteria included, for example, diagnoses of subarachnoid hemorrhage, traumatic brain injury (including traumatic intracranial hemorrhage), and spontaneous intracerebral hemorrhage. Patients that had a history of cardiac arrest and patients found to be in status epilepticus were excluded. From the electronic medical record (EMR) for each patient, medication dose administration records were obtained for the duration of the ICU admission and cataloged the exact time, dose, and route of administration of all antiepileptic medications. Continuous infusions of intravenous anesthetics were not included. As a continuous measure of level of consciousness, regularly sampled Glasgow Coma Scale (GCS) scores for the duration of the ICU admission were obtained from the medical record.

For each patient, the clinical report of the continuous EEG was reviewed. As above, patients found to be in electrographic status epilepticus (using ACNS criteria) were excluded. For the remainder of the patients, EEG findings were subsequently classified by the presence or absence of ictal interictal continuum (IIC) activity. EEG reports that indicated either a normal pattern of activity, polymorphic slowing, generalized rhythmic delta activity, or rare to occasional sporadic discharges were classified as IIC absent; EEG reports with lateralized rhythmic delta activity, frequent to continuous and/or periodic discharges, brief potentially ictal rhythmic discharges (BIRDs), stimulus-induced rhythmic/periodic/ictal discharges (SIRPIDs), or isolated seizures (but not meeting the criteria for status epilepticus) were classified as IIC present.

For this example, all cEEG recordings were acquired utilizing in a standard 10-20 electrode arrangement at either 256 or 512 Hz. Recordings were low-pass filtered at 125 Hz using a third-order Butterworth filter, notch filtered at 60 Hz and 120 Hz to reduce line noise, and referenced to the average. For each EEG in the data set, a series of quantitative parameters were calculated. For example, four frequency-based parameters (power in the alpha band, delta band, theta band, and alpha/delta ratio) and eight functional-connectivity based parameters (density, characteristic path length, clustering coefficient, size of largest component, number of independent components, number of non-trivial components, characteristic path length of largest component, and clustering coefficient of largest component) were calculated.

All quantitative metrics were calculated in 1000-ms intervals with 500-ms overlap between intervals. Power within the alpha, theta, and delta bands was normalized relative to the overall power in the EEG signal. To calculate the functional connectivity measures in this example, within a given 1000-ms segment, the signal at each electrode was z-normalized to zero mean and unit variance. A cross-correlogram of the EEG activity was calculated between all pairs of electrodes using a +/−250-ms sliding window of overlap. The peak in the cross-correlogram between each pair of electrodes was identified, and statistically significant peaks in the cross-correlation defined a connection. For each 1000-ms epoch of data, a network of nodes (the EEG electrodes) and edges (the statistically-significant cross-correlations defining the connections between them) may be calculated. From each functional connectivity network, the graph theoretical measures described above may be calculated. All EEG data may then be assembled into time series that were registered with time-stamped medication administration records and quantitative neurologic exam assessments (i.e. the GCS score) extracted from the EMR.

At the time of each medication administration (or, in the case of the control intervention, the time of each neurologic exam), in this example the twelve quantitative EEG (qEEG) metrics were examined for the 1 hour prior to medication administration to the 6 hours after medication administration. Based on the sign of the change in the GCS score 24 hours after the medication administration event, each event may then be assigned to one of three categories: decline, stable, or improve. Each qEEG curve may be normalized to the mean value of the parameter during the 1 hour prior to the medication administration event to evaluate change from baseline.

These three categories (decline, stable, and improve) may subsequently serve as the outcome labels for implementation of the machine learning algorithm. For each application described, a bootstrapping approach with 100 iterations may be employed. On each iteration, the data set in this example may be randomly split into a training data set (80% of the patients) and a testing data set (the remaining 20% of the patients). In this example, the mean values of each of the twelve qEEG metrics were calculated over the 5-hour time period encompassing 1 to 6 hours post-medication administration. These data served as the observations used to train a K-nearest neighbor (KNN) clustering algorithm, with the sign of the change in GCS at 24 hours as the outcome label. This KNN model may be trained on the training data (using 5-fold cross-validation) to assign EEGs to one of three predicted groups: decline, stable, or improve. As a control group, another KNN model may be fit using the same EEG data but with randomly shuffled outcome labels. Once trained, both models (the true model and control model) were then tested on the qEEG data from the 20% of the patients that had been withheld; the withheld qEEG data was thus assigned by the model a predicted clinical trajectory. Time-series curves showing the change in GCS score from the time of medication administration for each medication administration event in the testing data subset were clustered based on predicted class. In this example, the bootstrap process was repeated 100 times to generate mean+/−95% confidence interval (CI) curves for each cluster.

In this example, two statistical tests were performed to assess the performance of the various predictive models. In the first statistical test, to demonstrate that these models could predict outcome superior to chance, for each bootstrap iteration, the Spearman rank correlation was calculated between the predicted class (−1, 0, 1 for predicted to decline, remain stable, or improve, respectively) and the mean change in the GCS score relative to the time of medication administration over the 24-168 hours post-medication administration for each bootstrap iteration. Spearman rank correlation was also calculated between the class predicted by the control model and the mean change in GCS for each iteration. The means of these two distributions of correlation coefficients (derived from the true model and the control model) were then compared using a two-tailed Student's t-test.

As a second test, to assess predictive accuracy of the model, the sensitivity and specificity of each of the model's non-stationary predictions (decline, improve) was calculated and used them to calculate a positive likelihood ratio (sensitivity/(1−specificity)). For the “decline” predicted class, correct predictions were considered all those in which the mean change in GCS score from 24-168 hours was a greater than 1 point decline and for those in the “improve” class, correct predictions were all those in which the mean change in GCS score from 24-168 hours was a greater than 1 point improvement. Using these definitions for correct predictions, sensitivity, specificity, and positive likelihood ratios for the two non-stationary prediction classes were calculated. Bootstrapping with 100 iterations was used to generate a distribution of results with a mean and 95% CI. A model was considered to have statistically significant predictive power if the mean and lower bound of 95% CI of the likelihood ratio are both greater than 1.

In this example, 210 patients met initial inclusion criteria; 6 were excluded for clinical reasons (identified as being in status epilepticus during cEEG recording). Of the 204 patients (119 female, 85 male) included in the analysis, 140 had an admission diagnosis of subarachnoid hemorrhage, 49 had an admission diagnosis of traumatic brain injury (including traumatic intracranial hemorrhage), and 15 had a diagnosis of spontaneous intracerebral hemorrhage. Age ranged from 21 to 88 years (average 59.3 years, median 59.0 years).

Most patients were treated with at least one AED. A total of 5,748 doses of AEDs were administered. Of the 204 patients included in the analysis, 9 patients received no AEDs, 104 patients were treated with 1 AED, 66 patients were treated with 2 AEDs, 19 patients were treated with 3 AEDs, 5 patients were treated with 4 AEDs, and 1 patient was treated with 5 AEDs Medications received included, for example, clobazam, clonazepam, diazepam, phenytoin (including fosphenytoin), gabapentin, levetiracetam, oxcarbazepine, phenobarbital, topiramate, valproic acid, carbamazepine, lacosamide, and valproic acid.

When compared to the mean GCS over the prior 24 hours, in this example initiation of a new AED was associated with a mean 0.38-point improvement in GCS score during the 24 hours after initiation. As a control, the mean change GCS score was also calculated over the 24-hour period before and after each neurologic exam during which no AED was administered for 72 hours. Performance of this control intervention was associated with a 0.12-point improvement in GCS score. The increase in GCS score following initiation of a new AED was significantly greater than after a neurologic exam (p=8.80×10⁻⁸, two-tailed Student's t-test). As the rate of GCS score is expected to vary over the course of admission (e.g. a greater changes in GCS score are expected early in admission when clinical status is more unstable), this comparison was repeated within a specified time window of admission. When the time interval of comparison in this example was limited to 48 hours or less, undergoing a neurologic exam (while not receiving AED therapy) was associated with a greater subsequent 24-hour improvement in GCS score than occurred after starting an AED. There was no difference between mean GCS score in patients that did or did not receive an AED during this time interval. The relationship between AED regimen intensification and GCS score was also examined. In this example, initiating a first AED was followed by a 0.54-point average improvement in GCS score that was significantly greater than 0 (p=6.76×10⁻⁸, two-tailed Student's t-test); initiating a second, third, fourth, or fifth AED yielded no significant change in GCS. However, regression analysis revealed significant inverse relationship between mean GCS score change and AED number (β₁=−0.252, 95% CI: −0.4322, −0.0710).

As mentioned above in this example a series of qEEG parameters were calculated for each patient included in the analysis. Specifically, four frequency-based metrics (power in the alpha band, delta band, theta band, and alpha/delta ratio) and eight functional-connectivity based metrics (density, characteristic path length, clustering coefficient, size of largest component, number of independent components, number of non-trivial components, characteristic path length of largest component, and clustering coefficient of largest component) were calculated. All data were assembled into time series that were registered with time-stamped medication administration records and quantitative neurologic exam assessments (the GCS score) extracted from the EHR.

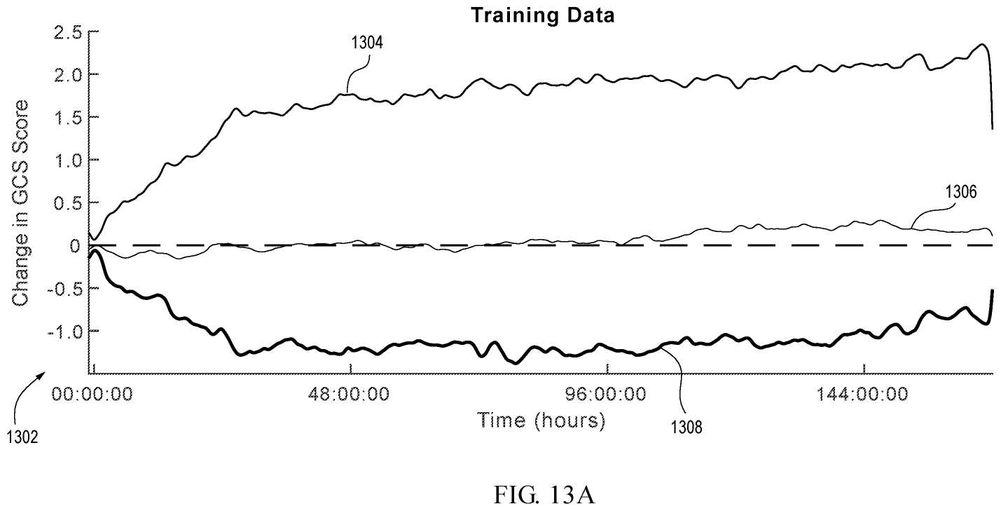

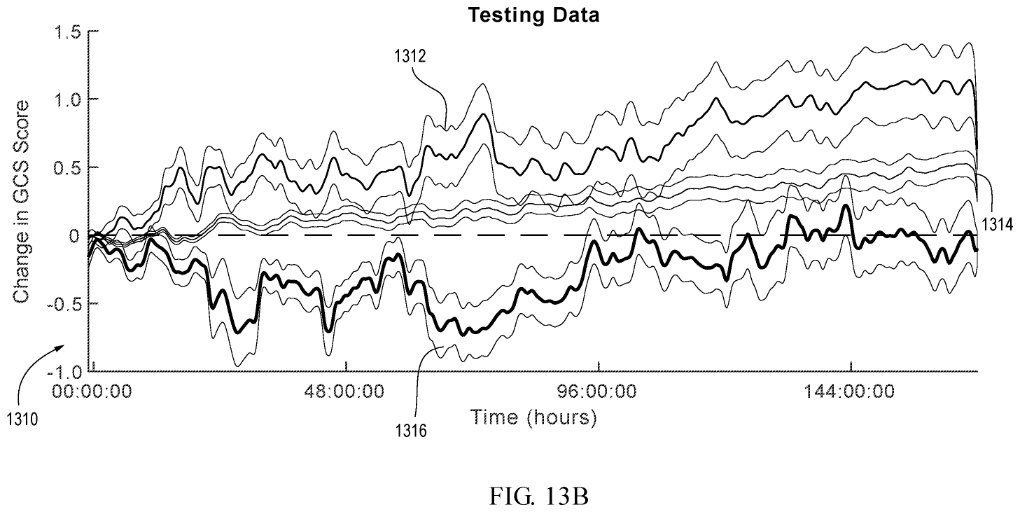

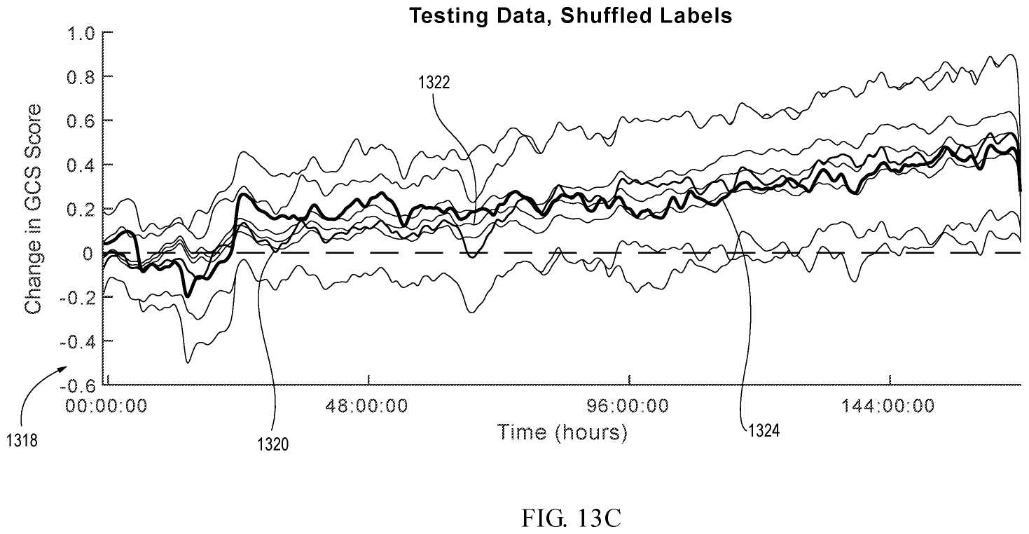

The changes in each of these 12 qEEG features were examined around the time of each AED dose administration. To determine whether EEG features could be used to predict the clinical response to AED treatment, a k-nearest neighbor (KNN) clustering algorithm was trained to classify segments of EEG data recorded immediately following AED dose administration into anticipated GCS score trajectory groups, predicting either (1) an improvement in GCS score, (2) a decline GCS score, or (3) no change in GCS score. The sign of the change in GCS score 24 hours after the administration of the AED was used as the outcome label. In this example, on each of 100 iterations qEEG data from 80% of the patients were randomly selected to train the clustering algorithm and the remaining 20% of the data were withheld for testing. FIG. 13A is a graph 1302 illustrating mean GCS score trajectories over 100 bootstrap iterations during training of a classifier with five-fold internal cross-validation in accordance with an embodiment. In FIG. 13A, curves derived from the KNN clustering algorithm during training for improved GCS score 1302, stable GCS score 1306, and declining GCS score 1308 are shown. After training with 5-fold internal cross-validation (shown in FIG. 13A), the withheld EEG was presented to the model for external validation and the clinical trajectory (improve, decline, no change) of the GCS score following the medication administration was predicted. The change in GCS scores over time following each medication administration event were then averaged within each cluster to generate three mean GCS trajectory curves. FIG. 13B is a graph 1310 illustrating mean GCS score trajectories generated by a trained classifier in accordance with an embodiment. In FIG. 13B, the mean GCS score trajectory curve for the GCS scores predicted by the trained KNN clustering algorithm for improved GCS score 1312, stable GCS score 1314 and declining GCS score 1316 are shown. In this example, patients whose qEEG features cluster most closely with those that had an early favorable response to AED treatment had a rise in GCS score over the 48 hours following dose administration that was subsequently sustained over the following 5 days and patients with qEEG features that cluster with those with an unfavorable response to treatment experience the opposite. In this example, patients with EEG features that cluster with those that demonstrated no change in GCS in response to AED treatment had only modest rise in GCS score over the subsequent week.

In this example, the performance of this model was quantified in two ways. To demonstrate superiority over chance, a control model was created by fitting the KNN algorithm using the same EEG data using randomly shuffled outcome labels. FIG. 13C is a graph 1318 illustrating mean GCS score trajectories for a control model for evaluating performance of a classifier in accordance with an embodiment. In FIG. 13C, the mean GCS score trajectory curve for the GCS scores predicted by the control model for improved GCS score 1320, stable GCS score 1322 and declining GCS score 1324 are shown For each set of model results (i.e. the predictions of the model when tested on the hold-out data set and the predictions of the model trained on shuffled outcome labels tested on the hold-out data set), for each of the 100 iterations of the bootstrapping process, the Spearman rank correlation between mean change in GCS score from 24 to 168 hours following AED dose administration and predicted GCS trajectory (1 for improved, 0 for stable, or −1 for worsened) was calculated. In this example, the model was said to have performed better than chance if the Spearman rank correlation from the hold-out data set predictions was greater than that from the shuffled label with a p-value of the two-tailed Student's t-test <0.05. As a second test of performance, the positive likelihood ratio (sensitivity/(1−specificity)) for the two non-stationary predictions (improve or decline) was calculated, again using bootstrapping with 100 iterations to generate 95% confidence intervals around the estimates. A model was considered to have statistically significant predictive power if the mean and lower bound of 95% CI of the likelihood ratio are both greater than 1.

Given the difference in pharmacokinetics between different routes of medication administration and the anticipated heterogeneity in EEG effects that this may produce, it was explored whether the performance of the predictive model could be improved by limiting the quantitative analysis to specific subset of the population. In this example, it was also explored whether there were differences in model performance when analysis was repeated using subsets of patients with specific range of GCS score, underlying diagnosis, route of medication administration, specific medication administered, number of doses previously administered, and whether the EEG demonstrated an IIC pattern of activity. Though in many instances reducing the sample size for the clustering algorithm to a subset of the population led either to a loss of statistical significance or to an inability for the machine learning algorithm to converge, the model retained performance better than chance in a number of instances.

To try and determine whether these effects were truly assessing the response to medication administration and not simply ongoing fluctuations in the EEG, the same machine learning algorithm was applied to epochs of quantitative EEG data recorded during regularly timed interventions (e.g. neurologic exams) during which no AEDs were administered for at least 72 hours. The change in GCS score 24 hours following the intervention (in this case, a documented neurologic exam) was again used as the outcome label for the KNN clustering algorithm, and as above, qEEG data from 80% of the patients were randomly selected as a training set and 20% as a testing set. The process was repeated over 100 iterations and the mean predicted GCS score trajectories for each of the three outcome clusters was determined. When trained on this dataset, the clustering algorithm performs no better than chance at predicting the trajectory of the GCS score, suggesting that this data set comprised of random epochs of EEG data removed in time from AED administration cannot similarly be used predict clinical course.

Computer-executable instructions for determining (or predicting) a treatment outcome for neurological disorders and injuries based on functional connectivity parameters according to the above-described methods may be stored on a form of computer readable media. Computer readable media includes volatile and nonvolatile, removable, and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital volatile disks (DVD) or other optical storage, magnetic cassettes, magnetic tape, magnetic disk storage or other magnetic storage devices, or any other medium which can be used to store the desired instructions and which may be accessed by a system (e.g., a computer), including by internet or other computer network form of access

The present invention has been described in terms of one or more preferred embodiments, and it should be appreciated that many equivalents, alternatives, variations, and modifications, aside from those expressly stated, are possible and within the scope of the invention.

1. A system for determining a treatment outcome for a subject for a neurological disorder, the system comprising:
a processor configured to receive a set of electroencephalogram (EEG) data associated with the subject and configured to determine at least one functional connectivity parameter based on the EEG data;
a classifier coupled to the processor and configured to receive the at least one functional connectivity parameter and configured to generate a prediction for a treatment outcome based on the at least one functional connectivity parameter; and
a display coupled to the classifier, the display configured to display the prediction for the treatment outcome.

2. The system according to claim 1, wherein the classifier is a supervised learning network.

3. The system according to claim 1, wherein the treatment outcome is a treatment outcome for an intravenous anesthesia weaning process.

4. The system according to claim 3, wherein the neurological disorder is refractory status epilepticus (RSE).

5. The system according to claim 3, wherein the classifier is a support vector machine (SVM).

6. The system according to claim 3, wherein the prediction for a treatment outcome generated by the classifier indicates whether the weaning process will be successful or unsuccessful.

7. The system according to claim 1, wherein the EEG data is continuous electroencephalography (cEEG) data.

8. The system according to claim 1, wherein the treatment outcome is a treatment outcome of the administration of an antiepileptic drug (AED) to the subject.

9. The system according to claim 8, wherein the neurological disorder is acute brain injury (ABI).

10. The system according to claim 8, wherein the classifier is a k-nearest neighbor (KNN) clustering model.

11. The system according to claim 8, wherein the prediction for a treatment outcome generated by the classifier indicates a classification of a change in a Glasgow Coma Scale (GCS) score over a predetermined period of time.

12. The system according to claim 8, wherein the processor is further configured to determine at least one frequency-based parameter based on the EEG data and the classifier is configured to generate a prediction for a treatment outcome based on the at least one functional connectivity parameter and the at least one frequency-base parameter.

13. A method for determining a treatment outcome for a subject for a neurological disorder, the method comprising:
acquiring a set of electroencephalogram (EEG) data from the subject;
determining, using a processor, at least one functional connectivity parameter based on the set of EEG data;
generating, using a classifier, a prediction for a treatment outcome based on the at least one functional connectivity parameter; and
displaying the prediction for the treatment outcome using a display.

14. The method according to claim 13, wherein the classifier is a supervised learning network.

15. The method according to claim 13, wherein the treatment outcome is a treatment outcome for an intravenous anesthesia weaning process.

16. The method according to claim 15, wherein the neurological disorder is refractory status epilepticus (RSE).

17. The method according to claim 15, wherein the prediction for a treatment outcome generated by the classifier indicates whether the weaning process will be successful or unsuccessful.

18. The method according to claim 13, wherein the treatment outcome is a treatment outcome of the administration of an antiepileptic drug (AED) to the subject.

19. The method according to claim 18, wherein the neurological disorder is acute brain injury (ABI).

20. The method according to claim 18, wherein the prediction for a treatment outcome generated by the classifier indicates a classification of a change in a Glasgow Coma Scale (GCS) score over a predetermined period of time.
